(19) United States
(12) Patent Application Publication
(10) Pub. No.: US 2006/0211965 A1
(43) Pub. Date: Sep. 21, 2006

(54) Device for the delivery of blood clotting materials to a wound site

(75) Inventors: Jeffrey L. Horn, Rocky Hill, CT (US); Raymond J. Huey, Orange, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-Medica, LLC, Wallingford, CT (US)

(21) Appl. No.: 11/082,320

(22) Filed: Mar. 16, 2005

(51) Int. Cl. A61F 5/00

(52) U.S. Cl. 602/13

(57) ABSTRACT

A sleeve for covering a portion of a limb at a wound site incorporates a molecular sieve material which is at least partially exposed so that blood flowing from a wound site over which the sleeve is positioned contacts the molecular sieve material to facilitate clotting. One end of the sleeve may be closed to form a stocking or a mitten. The molecular sieve material may also be incorporated into a cap or similar device for wearing on the head of a victim. A chin strap may be attached to the cap to facilitate the retaining of the cap on the victim's head. The molecular sieve material may also be incorporated into a bandage that is directly wearable on a wound.

### TECHNICAL FIELD

The present invention relates generally to blood clotting devices and, more particularly, to a device that allows blood clotting materials incorporated therein to be brought into contact with a wound site, particularly a head or limb wound. The present invention is also directed to methods for the use of such devices as bleeding control devices.

### BACKGROUND OF THE INVENTION

Blood is a liquid tissue that includes red cells, white cells, corpuscles, and platelets dispersed in a liquid phase. The liquid phase is referred to as plasma and includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances humans (as well as animals) can be wounded, thereby resulting in bleeding. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stop the flow of blood in a timely manner.

In an effort to address the above-described problems, materials and devices have been developed for controlling excessive bleeding in situations where conventional aid is unavailable or less than optimally effective. Although these materials and devices have been shown to be somewhat successful, they are sometimes not effective enough for traumatic wounds and tend to be expensive. Furthermore, these materials are sometimes ineffective and can be difficult to apply as well as remove from a wound.

In situations in which traumatic wounds are experienced (such as automobile or motorcycle accidents) and where multiple wounds are inflicted on a victim over multiple portions of the victim's body, some wounds may be inflicted on both the anterior and posterior surfaces of the victim. First aid may be administered to treat the wounds on one surface, but other wounds on another surface may be more difficult to attend to. For example, a victim's wounds on the front and sides of his legs may be treated while the victim is laying on his back, but the wounds on the back of his legs may be inaccessible because of the need to maintain the victim face up, particularly during transport of the victim to a medical facility. Furthermore, wounds on the posterior surfaces of a victim's arms, legs, and head may be completely overlooked during transport of the victim and may only become apparent upon closer inspection when the victim is moved from a gurney or stretcher to an operating table. Particularly with regard to lacerations, punctures, or scrapes on the posterior surfaces of the head or limbs that are hidden by the victim's hair or clothing, blood loss may be unnecessarily substantial before the wound is discovered. This problem is further exacerbated in cases where a victim is covered in blood and other debris as is sometimes the case in automobile accidents.

Based on the foregoing, it is the general object of the present invention to provide devices for controlling bleeding and methods of their use that overcome or improve upon the prior art.

### SUMMARY OF THE INVENTION

According to one aspect, the present invention resides in a sleeve for covering a portion of a limb at a wound site. As used herein, the word “sleeve” should be broadly construed to encompass tubular coverings that may be received over a person's upper or lower limb to cover part or substantially all of the person's limb. The sleeve incorporates a molecular sieve material which is at least partially exposed so that blood flowing from a wound site over which the sleeve is positioned contacts the molecular sieve material to facilitate clotting. Preferably, the molecular sieve material is retained in a mesh structure on an inner surface of the sleeve.

In another aspect of the present invention where a victim suffers an injury to a lower extremity, a stocking incorporating a molecular sieve material can be positioned over at least a portion of a foot and, optionally, over at least a portion of a leg. The stocking can have a closed end in which the foot is received. However, the end can also be open. For a foot wound, the stocking is donned over the foot of the victim. The molecular sieve material can also be retained in a mesh structure on an inner surface of the stocking and maintained in contact with the wound.

In still another aspect of the present invention, a mitten or glove incorporating a molecular sieve material can be positioned over a hand and, optionally, at least a portion of an arm of a victim suffering an injury to an upper extremity. The molecular sieve material is retained in a mesh structure on an inner surface of the mitten or glove and is maintained in contact with the wound.

In still another aspect of the present invention, molecular sieve material may be incorporated into a cap or similar device for wearing on the head of a victim. In such embodiments, the cap can be pulled over the crown of the head of the victim and adjusted as necessary to contact wounds on the back and/or sides of the scalp as well as the ears or back of the neck. A chin strap may be attached to the cap to facilitate the retaining of the cap on the victim's head. While the molecular sieve material has been described as being retained in a mesh, the present invention is not limited in this regard as the molecular sieve material can also be impregnated into portions of the wound treating devices described herein without departing from the broader aspects of the invention.

In still another aspect of the present invention, a bandage applicable to a bleeding wound may include a substrate, an adhesive disposed on a surface of the substrate, and particles of a molecular sieve material retained in the adhesive and partially extending therefrom. In such a bandage, the particles of molecular sieve material are arranged to be brought into contact with blood emanating from the wound when the bandage is applied to a wound site.

Surprisingly, bleeding wounds to the backs of a victim's arms, legs, and head are often missed by emergency services personnel when treating a victim with other injuries who is perhaps covered with blood or other debris from, for example, a motor vehicle accident. An advantage of the present invention is that wounds to the backs of a victim's arms, legs, or head would be treated to stop bleeding merely by placing the victim's arms and/or legs into the sleeves, stockings, or mittens of the present invention or by placing the cap of the present invention onto the victim's head. By employing the disclosed devices such that the wounds engage the zeolite (or other molecular sieve) material in the devices, clotting of blood emanating from the wound is facilitated regardless of whether or not the personnel treating the wound are aware of its existence.

Another advantage of the present invention is that the proper dose of molecular sieve material can be readily applied to an open wound. Particularly when the device is a sleeve or the like containing zeolite material, the device can be readily removed from sterilized packaging and used to treat the wounds directly at the points from which blood emanates to facilitate clotting of the blood without spilling powder or pellets outside the wound area. Guesswork, estimation, or calculation of the amounts of molecular sieve material for application to a bleeding wound is eliminated. Accordingly, little or no molecular sieve material is wasted.

### BRIEF DESCRIPTION OF THE DRAWINGS

FIG. 1 is a perspective view of a blood clotting sleeve of the present invention.

FIG. 2 is a front sectional view of the sleeve of FIG. 1.

FIG. 3 is a schematic representation of a permeable layer of the sleeve of FIG. 1.

FIG. 4 is a schematic representation of the interconnection of the permeable layer with the material of the outer surface of the sleeve.

FIG. 5 is a perspective view of a blood clotting sleeve incorporating tie straps along the lengths of the sleeve.

FIG. 6 is a perspective view of a blood clotting stocking of the present invention.

FIG. 7 is perspective view of a blood clotting mitten of the present invention.

FIG. 8 is a perspective view of a blood clotting cap of the present invention.

FIG. 9 is a front view of the cap of FIG. 8 showing a chin strap attached thereto.

FIG. 10 is a perspective view of a bandage having molecular sieve particles adhesively retained thereon.

FIG. 11 is a sectional view of the bandage of FIG. 10 showing the retaining of particles on the bandage.

### DETAILED DESCRIPTION OF THE PREFERRED EMBODIMENTS

Disclosed herein are devices and methods for delivering materials to wounds to promote the clotting of blood and the dressing of the wounds. The devices can be worn by victims to maintain contact between the devices and bleeding wound sites of the victim such that materials incorporated into the devices are contacted by the tissue of the wound to minimize or stop blood flow by absorbing at least portions of the liquid phases of the blood, thereby promoting clotting. The devices encompass sleeves, stockings, or mittens, each comprising a flexible tubular shell having a blood clotting agent disposed on at least a portion of an inner surface thereof. The devices also encompass caps that can be worn on the head to contact wounds on the scalp, ears, or nape of the neck, the caps having blood clotting agent disposed on at least a portion of an inner surface thereof. The devices also encompass bandages that can be disposed directly on wounds. In any device, the blood clotting agent is preferably a particulate molecular sieve material that can be maintained in direct contact with blood emanating from a wound through a permeable layer.

The molecular sieve material used in the present invention may be a synthetic polymer gel, cellulose material, porous silica gel, porous glass, alumina, hydroxyapatite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

The molecular sieve material is preferably a zeolite. Other molecular sieve materials that may be used include, but are not limited to, faujasite. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the zeolite is a friable material that is about 90% by weight calcium and about 10% by weight sodium. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, a polysaccharide, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), bacteriostatics, compounds containing silver ions, and the like. Other materials that can be incorporated to provide additional hemostatic functions include ascorbic acid, tranexamic acid, rutin, and thrombin. Botanical agents having desirable effects on the wound site may also be added.

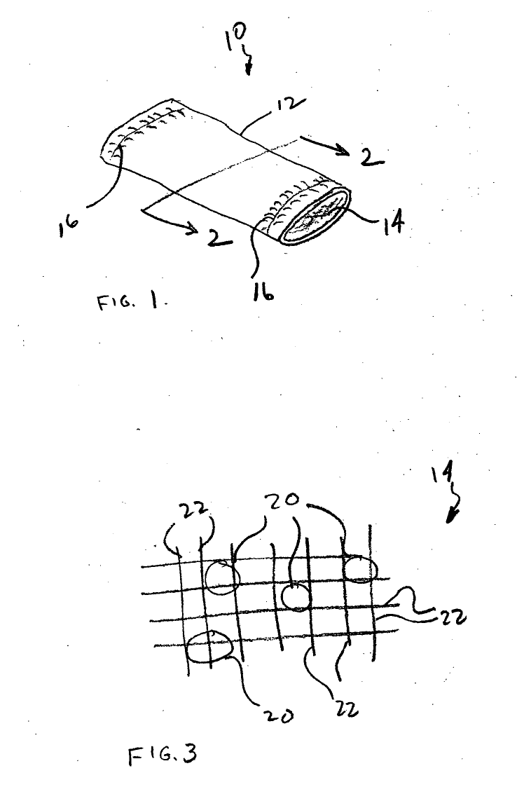

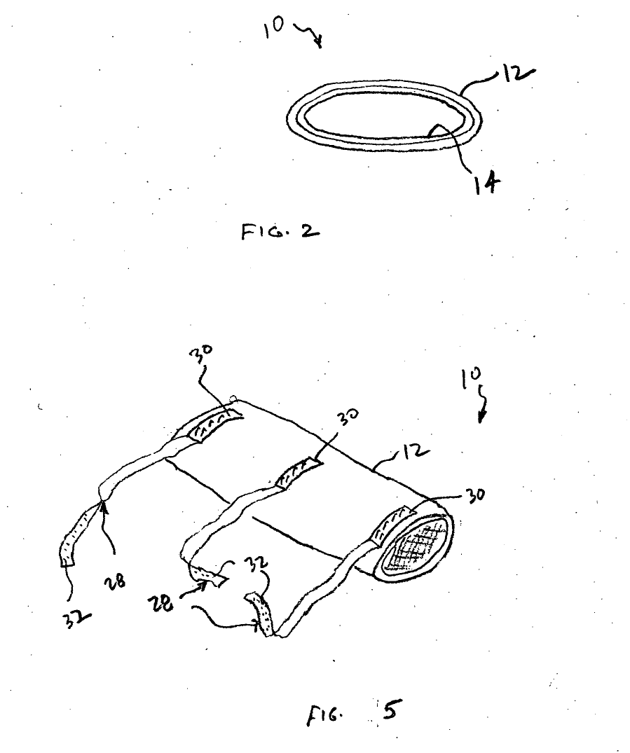

In one embodiment of the present invention shown in FIGS. 1 and 2, a sleeve that can be donned by a victim having a wound on an arm or a leg is shown at reference numeral 10. Blood clotting agents incorporated into an inner surface of the sleeve 10 are maintained in contact with the wound site to facilitate the clotting of blood directly at the wound site. The sleeve 10 is a flexible tubular shell 12 having a permeable layer 14 attached to an inner surface of the shell. The inner surface of the tubular shell 12 may comprise an insulating material such as a polyester/nylon blend. The permeable layer 14 is attached directly to the inner surface of the tubular shell 12 or to the insulating material using any suitable method such as, for example, stitching, gluing, combinations of the foregoing, and the like to form a receptacle. In a preferred embodiment, the tubular shell 12 is an elastic knit or weave that is expandable to allow pressure to be maintained substantially uniformly over the entire length and over the entire circumference of the cross section of the shell. When the sleeve 10 is worn by a victim to cover a bleeding wound, the permeable layer 12 allows blood to pass through therethrough and contact blood clotting zeolite (or other molecular sieve) material retained in the layer. Elastic members 16 may also be sewn into the tubular shell 12 proximate or adjacent the openings thereof to facilitate the retaining of the sleeve 10 on the victim.

Referring now to FIG. 3, the permeable layer 14 includes openings that are capable of retaining the zeolite particles 20 therein while allowing blood to flow through. As illustrated, the permeable layer 14 is shown as a mesh material, and only a few zeolite particles 20 are shown. Sealed packaging (not shown) provides a sterile environment for storing the sleeve 10 until it can be used.

The permeable layer 14 is defined by interconnected strands, filaments, or strips of material. The strands 22 can be interconnected in any one or a combination of manners including, but not limited to, being woven into a gauze, intertwined, integrally-formed, and the like. Preferably, the interconnection is such that the permeable layer 14 can flex to conform to the shape of a wound and to retain the shape of the wound while substantially maintaining the dimensions of the openings defined by the interconnection of the strands 22. The material from which the strands 22 are fabricated may be a polymer (e.g., nylon, polyethylene, polypropylene, polyester, or the like), metal, fiberglass, or an organic substance (e.g., cotton, wool, silk, or the like).

The openings defined by the permeable layer 14 are dimensioned to retain the zeolite particles 20 but to accommodate the flow of blood therethrough. Because the zeolite particles 20 may be tightly packed into the permeable layer 14, the particles may partially extend through the openings. However, it is not a requirement of the present invention that the zeolite particles 20 protrude through the openings of the permeable layer 14. If the zeolite particles 20 do extend through the openings, the particles are able to directly contact tissue to which the sleeve is applied. Thus, blood emanating from the tissue immediately contacts the zeolite particles 20, and the water phase thereof is wicked into the zeolite material, thereby facilitating the clotting of the blood.

The zeolite particles 20 are substantially spherical or irregular in shape (e.g., balls, beads, pellets, or the like) and about 0.2 millimeters (mm) to about 10 mm in diameter, preferably about 1 mm to about 7 mm in diameter, and more preferably about 2 mm to about 5 mm in diameter. In any embodiment (balls, beads, pellets, etc.), less particle surface area is available to be contacted by blood as the particle size is increased. Therefore, the rate of clotting can be controlled by varying the particle size. Furthermore, the adsorption of moisture (which also has an effect on the exothermic effects of the zeolite) can also be controlled.

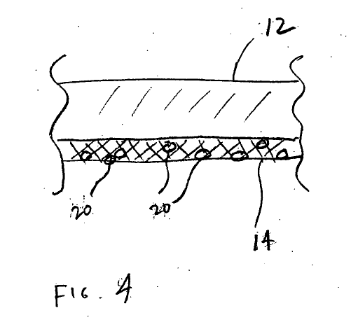

Referring to FIG. 4, the permeable layer 14 is positioned adjacent the tubular shell 12 of the sleeve 10. The tubular shell 12 preferably comprises an elastic material, as stated above, to which the permeable layer 14 is stitched, glued, or otherwise attached. As described above, the permeable layer 14 is defined by interconnected strands, filaments, or strips of material to form a woven gauze or the like. The strands, filaments, or strips of material are woven or otherwise arranged to define openings that are sized to retain zeolite particles 20 in the permeable layer 14.

Referring to FIG. 5, the sleeve 10 may be securable to the arm or leg of a victim via one or more straps 28. Portions of the straps 28 may be affixed to the outer surface of the tubular shell 12 at various points along the length of the sleeve 10 such that belt portions of the straps 28 may be wrapped around the sleeve and secured upon themselves. The straps 28 may be secured to themselves using hook-and-loop material, with one of the hook portion 30 and the loop portion 32 being attached to the ends of the straps secured to the outer surface of the tubular shell 12 and the other of the hook portion and the loop portion being disposed along the lengths of the belt portions. Alternately, the straps 28 may be nylon webbing or similar material, adjustable lengthwise using buckles, and securable to themselves using quick-disconnect fasteners.

Referring now to FIGS. 1-5, to utilize the sleeve 10 to dress a bleeding wound, the sleeve is removed from the packaging (if any) and held such that one end of the tubular shell 12 is opened. The sleeve 10 is then pulled over the victim's leg or arm such that the wound is covered. If the sleeve 10 has retaining straps 28 attached to the outer surfaces of the tubular shell 12, the straps may be wrapped around the tubular shell and secured upon themselves. Once positioned on the victim, the zeolite particles 20 in the permeable layer 14 contact the tissue of the wound and/or the blood, and at least a portion of the liquid phase of the blood is adsorbed by the zeolite material, thereby promoting the clotting of the blood.

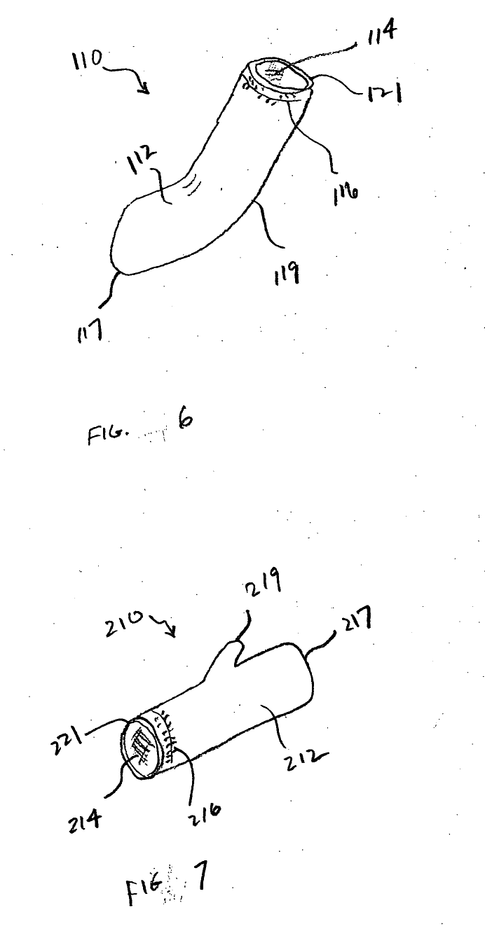

Another embodiment of the present invention is shown with reference to FIG. 6. In FIG. 6, a blood clotting stocking is shown generally at 110. Blood clotting stocking 110 comprises a tubular shell 112 having an outer shell and a permeable layer 114 attached to an inner surface of the tubular shell 112. A first end 117 of the tubular shell 112 is closed. The tubular shell 112 is configured to define a bend 119 intermediate the first end 117 and a second end 121. When donned by a victim having a foot wound, the victim's toes are positioned in the first end 117 and the victim's heel is substantially positioned in the bend 119. An elastic member 116 (or a strap or similar device) can be incorporated into the stocking 110 proximate the second end 121 to hold the stocking on the victim's calf or leg. As above, the permeable layer 114 comprises a mesh structure in which zeolite (or other molecular sieve materials) is maintained.

Still another embodiment of the present invention is shown with reference to FIG. 7. In FIG. 7, a blood clotting mitten is shown generally at 210. Blood clotting mitten 210 comprises a tubular shell 212 having an outer shell and a permeable layer 214 attached to an inner surface thereof. A first end 217 of the tubular shell 212 is closed to provide a pocket in which a victim's fingers can be positioned. A second end 221 of the tubular shell 212 is open to receive the victim's hand. An appendage 219 may extend from an intermediate portion of the tubular shell 212 to accommodate the victim's thumb. An elastic member 216 (or a strap or similar device) can be incorporated into the mitten 210 proximate the second end 221 to hold the mitten on the victim's wrist or arm. As above, the permeable layer 214 comprises a mesh structure in which zeolite (or other molecular sieve materials) is maintained.

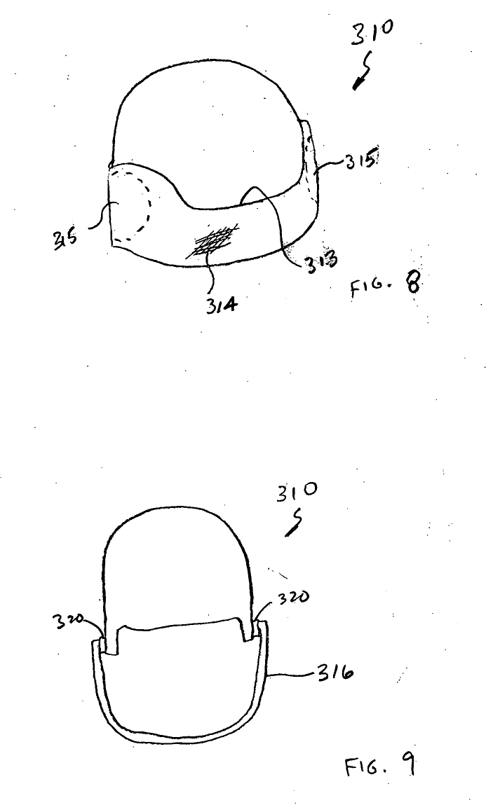

Referring now to FIG. 8, a device for delivering molecular sieve material to a head wound may be a cap 310. Cap 310 is configured to fit over the crown of a victim's head and to conform to the contours of the skull. A permeable layer 314 comprising a mesh structure in which zeolite (or other molecular sieve materials) is maintained is attached to an inner surface of the cap 310. The material of the outer shell of the cap 310 is preferably elastic (e.g., a weave, a knit, or stretchable) to allow the cap to be held against the head. Exemplary materials for the outer shell include, but are not limited to, wool, rubber, and the like. Other materials having limited elasticity (cotton, silk, and the like) may also be used in conjunction with elastics or similar devices attached to portions of the cap 310 to hold the cap on the victim's head. A lower edge 313 of the cap 310 may be foldable back over the cap to cover only a portion of the head. Ear flaps 315 may be integrally formed with or attached to the sides of the cap 310 to be folded down onto a victim's ears.

As is shown in FIG. 9, a chin strap 316 may be attached to the cap 310 to retain the cap on a victim's head. In particular, the chin strap 316 may be removably attached to the sides of the cap 310 proximate the sides (or the ear flaps) and fittable under the victim's chin. The chin strap 316 is preferably attached to the cap 310 using hook-and-loop material 320. One of the hook material and the loop material is preferably disposed along a length of the chin strap 316 to facilitate the adjustment of the strap on victims of different sizes. The chin strap 316 is particularly useful in embodiments in which the material of the cap 310 is substantially inelastic or in instances in which the victim is prone to removing the cap. The chin strap 316 may be padded along portions thereof to provide comfort to the victim while wearing the cap 310.

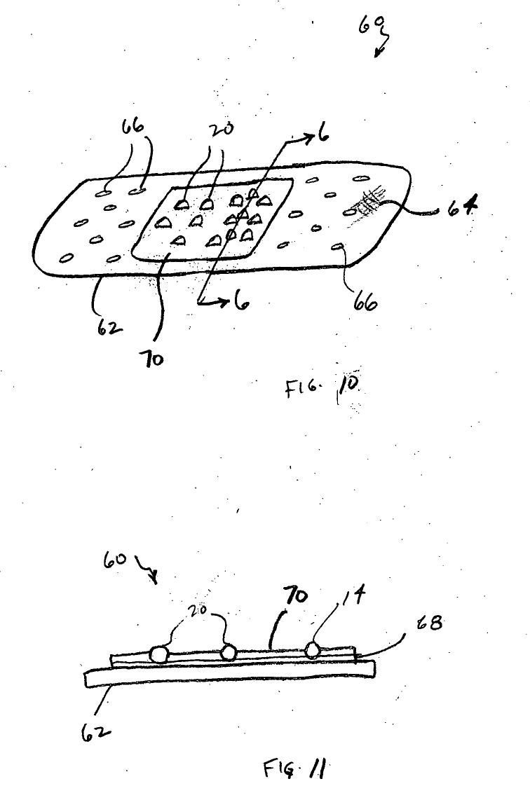

Referring now to FIG. 10, another embodiment of the present invention is a bandage, shown at 60, which comprises zeolite particles 20 (or some other molecular sieve material) adhesively mounted to a flexible substrate 62 that can be applied to a wound. The zeolite particles 20 are embedded in, for example, a first adhesive material 70 disposed on a wound-engaging surface of the bandage 60. The first adhesive material 70 may be any type of adhesive that is bio-compatible with the tissue of a wound and that is capable of retaining zeolite material therein, particularly in the moist environment of a bleeding wound. The zeolite particles 20 may be pellets, granules, beads, rods, flakes, chips, or the like, as well as combinations of the foregoing.

The substrate 62 is a plastic or cloth member that is conducive to being retained on the skin proximate a bleeding wound. A second adhesive material 64 (e.g., a pressure-sensitive adhesive) is preferably disposed on a surface of the substrate 62 to adhesively retain the bandage 60 on the skin. If the substrate 62 is a non-breathable plastic material, the substrate may include holes 66 to allow for the dissipation of moisture evaporating from the skin surface.

Referring now to FIG. 11, the zeolite particles 20 are embedded in the first adhesive material 70 to such a depth so as to allow the zeolite particles to protrude above the uppermost surface of the first adhesive material to contact the wound. An absorbent material 68 may form a layer between the substrate 62 and the first adhesive material 70. The absorbent material 68 provides a moisture barrier that prevents fluid from the blood or the blood itself from wicking into the substrate 62. The absorbent material 68 may also prevent fluids (e.g., perspiration, foreign blood, and the like) from being transferred through the substrate 62, into the zeolite particles 20, and into the wound.

In the preparation of zeolite material (i.e., formation of the material into particle form) for the devices of the present invention, an initial level of hydration of the zeolite may be controlled by the application of heat to the zeolite material either before or after the material is formed into particles. However, it has also surprisingly been found that as the particle size of the zeolite is increased, the moisture content has less of a correlative effect on any exothermia produced as the result of mixing the particlized zeolite in blood. As such, formation of the zeolite material into the zeolite particles may be by extrusion, milling, casting, or the like.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the above description.

What is claimed is:

1. An apparatus for promoting the clotting of blood, comprising:
a sleeve having an open end into which a limb can be inserted; and
a permeable layer for retaining molecular sieve material in particulate form therein attached to an inner surface of said sleeve, at least a portion of said permeable layer being defined by a mesh having openings therein;
wherein when treating a bleeding wound, donning said apparatus causes at least a portion of said particulate molecular sieve material to come into contact with blood through said openings.

2. The apparatus of claim 1, wherein said molecular sieve material is a zeolite.

3. The apparatus of claim 2, wherein said zeolite comprises particles having diameters of about 0.2 mm to about 10 mm.

4. The apparatus of claim 1, wherein said mesh is flexible.

5. The apparatus of claim 1, wherein said mesh is conformable to a shape of a wound and can retain said shape of said wound.

6. The apparatus of claim 1, wherein at least one of said particles of said particulate molecular sieve material protrudes through one of said openings.

7. The apparatus of claim 1, wherein said sleeve comprises a woven material that is expandable to allow pressure to be maintained substantially uniformly over a length of said sleeve and over a circumference of a cross section of said sleeve.

8. The apparatus of claim 1, wherein said sleeve comprises elastic members attached thereto to facilitate the retaining of said sleeve on said limb.

9. The apparatus of claim 1, wherein said sleeve comprises straps attached thereto to facilitate the retaining of said sleeve on said limb.

10. The apparatus of claim 1, wherein said sleeve comprises a closed second end.

11. The apparatus of claim 10, wherein said apparatus is configured at said closed second end as a mitten.

12. The apparatus of claim 10, wherein said apparatus is configured at said closed second end as a stocking.

13. A cap wearable on the head of a person for controlling bleeding on the head, said cap comprising:
a shell; and
a permeable layer for retaining molecular sieve material in particulate form therein attached to an inner surface of said shell, at least a portion of said permeable layer being defined by a mesh having openings therein;
wherein when treating a bleeding wound, donning said cap causes at least a portion of said particulate molecular sieve material to come into contact with blood through said openings.

14. The cap of claim 13, wherein said molecular sieve material is a zeolite.

15. The cap of claim 14, wherein said zeolite comprises particles having diameters of about 0.2 mm to about 10 mm.

16. The cap of claim 13, wherein said mesh is flexible.

17. The cap of claim 13, wherein said mesh is conformable to a shape of a wound and can retain said shape of said wound.

18. The cap of claim 13, wherein said shell comprises an elastic material that facilitates the retaining of said cap on the head.

19. The cap of claim 13, further comprising a chin strap attached to said shell, said chin strap being fittable under the chin of the person wearing said cap.

20. The cap of claim 13, wherein said shell further comprises ear flaps extending from the sides of said shell.

21. A bandage applicable to a bleeding wound, said bandage comprising:
a substrate;
a first adhesive disposed on a surface of said substrate; and
particles of a molecular sieve material retained in said first adhesive and partially extending therefrom, said particles of said molecular sieve material being arranged to be placed on a bleeding wound.

22. The bandage of claim 21, further comprising a second adhesive disposed on said surface of said substrate, said second adhesive being capable of adhering said bandage to skin adjacent said bleeding wound.

23. The bandage of claim 21, wherein said molecular sieve material is a zeolite.

24. The bandage of claim 23, wherein said zeolite has a form selected from the group consisting of pellets, rods, granules, beads, chips, flakes, and combinations of the foregoing forms.

25. The bandage of claim 21, further comprising an absorbent material between said substrate and said first adhesive.

26. A bandage applicable to a bleeding wound, said bandage comprising:
a substrate; and
particles of a molecular sieve material embedded in said substrate, said particles being at least partially exposed so that when said bandage is applied to a bleeding wound, said at least partially exposed particles come in contact therewith.
